(19) United States
(12) Patent Application Publication
(10) Pub. No.: US 2018/0113123 A1
(43) Pub. Date: Apr. 26, 2018

(54) SHRINK ELECTRODE

(71) Applicant: The Regents of the University of California, Oakland, CA (US)

(72) Inventors: Michelle Khine, Irvine, CA (US); Jonathan Pegan, Irvine, CA (US); Jason Heikenfeld, Cincinnati, OH (US); Adam Hauke, Cincinnati, OH (US); Kevin Plaxco, Santa Barbara, CA (US)

(21) Appl. No.: 15/789,833

(22) Filed: Oct. 20, 2017

Related U.S. Application Data
(60) Provisional application No. 62/410,741, filed on Oct. 20, 2016.

(51) Int. Cl. G01N 33/543; G01N 27/327

(52) U.S. Cl. CPC G01N 33/5438; G01N 27/3277

(57) ABSTRACT

A sensor including a flexible substrate, a conductor disposed on the flexible substrate, and a hydrophilic surface coating disposed on the conductor. The flexible substrate and the conductor form wrinkle as a result of the substrate being shrunk. The hydrophilic surface coating is disposed in, e.g., fills, the wrinkles or covers surface areas of the conductor within invaginations of the wrinkles. Also disclosed are methods of preparing the sensor and methods of detecting an amount of an analyte in an aqueous solution. Methods of detecting an amount of analyte can include contacting the sensor with an aqueous solution, and detecting an electrical signal with the sensor, wherein the electrical signal is indicative of the amount of the analyte.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57.

### BACKGROUND

### Field of the Invention

This application is directed to a simple, low-cost method to create rough, high surface area electrodes with a dissolvable polymer coating. When functionalized with aptamers, the electrodes can be used for target specific electrochemical sensing.

### REFERENCE TO SEQUENCE LISTING

The present application is being filed along with a Sequence Listing in electronic format. The Sequence Listing is provided as a file entitled 26921503_1.TXT, created Oct. 20, 2017, which is 117 Kb in size. The information in the electronic format of the Sequence Listing is incorporated herein by reference in its entirety.

### Description of the Related Art

Growth in the popularity of point-of-care and wearable devices has ignited significant research into miniaturized electrochemical sensor technologies (Bandodkar, A. J., Wang, J., 2014. Non-invasive wearable electrochemical sensors: as review. Trends Biotechnol. 32, 363-371; Kimmel, D. W., LeBlanc, G., Meschievitz, M. E., Cliffel, D. E., 2012. Electrochemical sensors and biosensors. Anal. Chem. 84, 685-707; and Heikenfeld, J., 2016. Non-invasive analyte access and sensing through eccrine sweat: challenges and outlook circa 2016. Electroanalysis 28, 1242-1249). The attraction to this class of sensors for such applications is due to their capabilities of being reagentless, single step, wash free, and reversible. Electrochemical sensors are not, however, without potentially important limitations. As with any electronic device, designers are confronted with signal-to-noise constraints, a problem that only multiplies with miniaturization (less signal), increased background noise due to less-sophisticated portable electronics, and increased electromagnetic interference faced for portable and wearable devices. Researchers in this field will thus need to develop novel innovations to solve the signal-to-noise ratio problem for miniaturized sensors.

An electrochemical sensor's performance is highly dependent on the surface area of its working electrode that interfaces with the sample, rendering their miniaturization problematic. Research in the use of “high surface area” electrodes presents a clever solution to this problem in which one creates a rough micro- and/or nanostructured surface topography that considerably enhances the working electrode's electrochemically active surface area (EASA) relative to its macroscopic footprint. While complex chemical deposition methods to this end have been available for decades (Salvarezza, R. C., Alonso, C., Vara, J. M., Albano, E., Martin, H. O., Arvia, A. J., 1990. Monte Carlo simulation applicable to the growth of rough metal overlayers: parametric relationships related to the electrochemical roughening. Phys. Rev. B: Condens. Matter Mater. Phys. 41, 502-512; Gabardo, C. M., Adams-McGavin, R. C., Vanderfleet, O. M., Soleymani, L., 2015. Rapid prototyping of microfluidic devices with integrated wrinkled gold micro-nano textured electrodes for electrochemical analysis. Analyst 140, 5781-5788; Sonney, S., Shek, N., Moran-Mirabal, J. M., 2015. Rapid bench-top fabrication of poly(dimethylsiloxane), polystyrene microfluidic devices incorporating high-surfacearea sensing electrodes. Biomicrofluidics 9, 026501), recent research has shown that enhanced EASA can be achieved more simply by heat-shrinking a polymer substrate coated with metal thin film electrodes (Gabardo, C., Zhu, Y., Soleymani, L., Moran-Mirabal, J. M., 2013. Bench-top fabrication of hierarchically structured high surface-area electrodes. Adv. Funct. Mater. 23, 3030-3039; Pegan, J. D., Ho, A. Y., Bachman, M., Khine, M., 2013. Flexible shrink-induced high surface area electrodes for electrochemiluminescent sensing. Lab Chip 13, 4205-4209). Fabrication of these shrink electrodes is low-cost and does not require sophisticated equipment or clean-room facilities.

The benchmark EASA improvement for shrink electrodes was set by Pegan et al., who observed a 6.6-fold increase in electrochemical signal per macroscopic unit area for such electrodes (Pegan, J. D., Ho, A. Y., Bachman, M., Khine, M., 2013. Flexible shrink-induced high surface area electrodes for electrochemiluminescent sensing. Lab Chip 13, 4205-4209). Alone, this significant result is evidence of the potential shrink electrodes offer lab-on-chip (LOC) and point-of-care (POC) applications, but has their full potential been reached? For these polyolefin substrate devices, shrinking reduces the footprint area of the gold film electrodes by about a factor of 20. The EASA enhancements associated with shrinking should thus theoretically also reach 20-fold. While constraints such as “secondary wrinkling” (Gabardo, C., Zhu, Y., Soleymani, L., Moran-Mirabal, J. M., 2013. Bench-top fabrication of hierarchically structured high surface-area electrodes. Adv. Funct. Mater. 23, 3030-3039) exist that prevent these first-generation devices from reaching the theoretical EASA limit, the substantial gap between demonstrated results and theoretical predictions poses a looming question as to whether the full advantage of the surface topography is being exploited.

Poor wettability leads to non-ideal EASA gains when air bubbles trapped in the wrinkled structure prevent the sample solution from accessing the maximum available surface area (Freschauf, L. R., McLane, J., Sharma, H., Khine, M., 2012. Shrink-induced superhydrophobic and antibacterial surfaces in consumer plastics. PLoS One 7, e40987, 2012).

### SUMMARY

We present here a superwetting (Drelich, J., Chibowski, E., 2010. Superhydrophilic and superwetting surfaces: definition and mechanisms of control. Langmuir 26, 18621-18623), surface modification via the addition of a dissolvable polymer coating (see FIG. 1b), which extends shrink electrode EASA enhancement by 2-fold. Using such modified shrink electrodes to fabricate electrochemical aptamer-based (E-AB) sensors, we find not only greatly improved signal-to-noise ratios, but also a large and unexpected increase in their signal gain (the change in relative signal upon binding saturating target). For example, we employed a kanamycin aptamer into the sensor platform that, in addition to the EASA enhancement, also exhibited a 2.2-fold signal gain improvement over the same E-AB sensors deployed on traditional electrodes. This work provides insights into wetting of structured surfaces and provides a new best-in-class approach for shrink electrode sensors.

Some embodiments relate to a sensor. In some embodiments, the sensor includes a flexible substrate. In some embodiments, the sensor includes a conductor disposed on the flexible substrate. In some embodiments, the sensor includes a hydrophilic surface coating disposed on the conductor. In some embodiments, the flexible substrate and the conductor form wrinkles as a result of the substrate being shrunk, and the hydrophilic surface coating fills the wrinkles or covers surface areas of the conductor within invaginations of the wrinkles.

In some embodiments, the flexible substrate is a polyolefin film.

In some embodiments, the conductor is a metal.

In some embodiments, the metal is gold.

In some embodiments, the conductor includes one-dimensional (1D) nano structures.

In some embodiments, the one-dimensional (1D) nanostructures are nanotubes.

In some embodiments, the hydrophilic surface coating is a hydrophilic polymer.

In some embodiments, the hydrophilic surface coating is a natural aqueous soluble polymer.

In some embodiments, the naturally occurring aqueous soluble polymer is a protein.

In some embodiments, the hydrophilic surface coating is a synthetic aqueous soluble polymer.

In some embodiments, the synthetic aqueous soluble polymer is polyvinyl pyrolidone (PVP).

In some embodiments the sensor further includes an aptamer bound to the sensor.

In some embodiments, the aptamer is specific to an aminoglycoside antibiotic.

In some embodiments, the aptamer is specific to kanamycin.

In some embodiments, the aptamer is SEQ ID NO: 1.

In some embodiments, the wrinkles include micro-scale invaginations.

Some embodiments relate to a method of preparing a sensor as disclosed herein. In some embodiments, the method includes a step of obtaining a flexible substrate. In some embodiments, the method includes depositing a conductive layer onto the flexible substrate. In some embodiments, the method includes shrinking the flexible substrate with the deposited conductive layer, wherein the flexible substrate and the deposited conductive layer forms wrinkles. Some embodiments include a step of filling the wrinkles formed by the flexible substrate and the conductive layer with a layer of an aqueous-soluble polymer.

In some embodiments, the aqueous-soluble polymer is applied to the shrunken flexible substrate in an aqueous solution comprising the aqueous-soluble polymer and a surfactant.

In some embodiments, the shrunken flexible substrate is placed in a vacuum to remove air bubbles.

Some embodiments relate to a method of detecting an amount of an analyte in an aqueous solution. In some embodiments, the method includes obtaining a sensor as disclosed herein. In some embodiments, the method includes a step of contacting the sensor with the aqueous solution. In some embodiments, the method includes a step of detecting an electrical signal with the sensor, wherein the electrical signal is indicative of the amount of the analyte.

### BRIEF DESCRIPTION OF THE DRAWINGS

FIG. 1 includes cross sectional schematic depictions of EASA optimization strategy (a) bare gold (Au) surface on heat-shrunk polyolefin (PO); (b) a gold surface with a poly-vinyl pyrolidone (PVP) coating. Schematics are not to scale.

FIG. 2 includes photographs of shrink electrodes (a) shrink electrode array after polytetrafluoroethylene (PTFE) coating and shrinking; (b) close up of shrunken bare gold electrode surface, and (c) close up of same shrunken gold electrode in (b) with added polyvinyl pyrolidone (PVP) coating.

FIG. 3 is a cyclic voltammagram comparison of bulk gold (0.79 mm² footprint) and polyvinyl pyrolidone (PVP) coated shrink electrodes (0.31 mm² footprint).

FIG. 4 Illustrates an electrochemical aptamer based (E-AB) sensor for kanamycin fabricated on a shrink electrode. Graph (a) shows square wave voltammagrams of the sensor in response to increasing amounts of kanamycin. Graph (b) shows a plot of signal gain versus target concentration (curve i) fits the Langmuir isotherm and is compared to the curve of a similar E-AB sensor fabricated on a traditional electrode (curve ii).

### DETAILED DESCRIPTION

### Electrochemical Sensors

In some embodiments, an electrochemical sensor apparatus is provided that includes a flexible substrate and a conductor. The conductor may initially be formed as a thin metal film, which is thereafter crumpled or wrinkled because the material it is formed upon is shrunk to a fraction of its initial size. A plurality of electrical contacts may be in electrical communication with the conductor. The electrical contacts can be disposed at one or more conductive region(s). In some embodiments, a plurality of contacts is provided and the contacts are disposed along the length of an elongate conductive region on the flexible substrate. The contacts in such an embodiment may be disposed to one side of the elongate conductive region and may allow connection to other devices at a number of different positions and/or permit a number of different devices to be in contact with the conductive region. The contacts can be used to measure a signal such as current.

Cross-sections of the wrinkled metal films reveal many tens of micron-scale invaginations in the surface where adjacent wrinkles are packed or disposed closely enough that they begin to coalesce, referred to as secondary folding.

### Method of Forming a Film Conductor

A micron-scale configuration can be provided by any suitable method. One technique involves exploiting a heat-shrink material. A polyolefin or a polystyrene shrink film can be masked and a metal thin film deposited thereon. The mask is removed and the shrink film is heated, e.g., to 160° C., shrinking the metal patterned polymer by about 67% by surface area. A flexible polymer, such as ECOFLEX30™, may be spin coated onto the shrunken sample and cured. A series of solvent baths, or other separation techniques, are used to lift off the polystyrene, resulting in the wrinkled metal thin film transferred onto the silicon elastomer. In some embodiments, a polymeric sheet of suitable heat-shrink characteristics is placed adjacent to a mask configured to block regions of the polymeric sheet. This may be followed by a step of depositing a conductive structure on the polymeric sheet at regions exposed through the mask. After the conductive structure is formed, the mask can be removed. The process then follows with shrinking the polymeric sheet with the conductive structure patterned on its surface by heating. The metal-patterned polymer may be reduced in size with regard to surface area by 5%, 10%, 15%, 20%, 25%, 30%, 35%, 40%, 45%, 50%, 55%, 60%, 65%, 70%, 75%, 80%, 85%, 90% or 95%. Thereafter, the conductive structure may be transferred to a flexible substrate.

The conductive structure can be deposited by any method, for example by air brushing or by electrospray of a material onto a surface. In some embodiments, the conductive structure comprises any conductive metal. In some embodiments, the metal conductive structure is a thin metal film. In some embodiments the metal is selected from the group consisting of Cu, Ag, Au, and Pt. In some embodiments, the polymeric sheet may be a shape-memory (e.g., a shrink-wrap) polyolefin (PO) film. The shrinking step may performed at a temperature of about 100° C., 110° C., 120° C., 130° C., 140° C., 150° C., 160° C., 170° C., 180° C., 190° C., 200° C., 210° C. or 250° C. Among the materials that are well suited for heat-shrink processing is polystyrene.

### Sensors Having a One Dimension Nanostructure

In some embodiments the sensor apparatus includes one-dimensional (1D) nanostructures. Such apparatus can include one or more of nanotubes, nanofibers, nanowires, and/or rods. A class of nanostructures includes nanoconductors. A nanostructure is said to be one dimensional, for example, if it is much longer in one direction than in other directions perpendicular to the long direction, for example having a diameter on the order of a nanometer (10⁻⁹ meters) and a length larger than 10 nm, larger than 50 nm, larger than 80 nm, larger than 90 nm or larger than 100 nm. Nanotubes include carbon nanotubes, for example. A nanowire is a nanostructure, with the diameter of the order of a nanometer (10⁻⁹ meters). A nanostructure can be defined as the ratio of the length to width being greater than 1000. Many different types of nanowires exist, including superconducting (e.g., YBCO), metallic (e.g., Ni, Pt, Au), semiconducting (e.g., Si, InP, GaN, etc.), and insulating (e.g., SiO₂, TiO₂). As disclosed herein, a 1D nanostructure is densified and aligned to produce an effective conductor, which may be configured as a thin film.

Various technologies provide a process to highly densify and align 1D nanostructures, such as CNTs, to improve its conductivity using shrink technology. In some embodiments, this is done by depositing a thin film of CNTs on the surface of a shape memory polymer, such as polyolefin. Preferably, the polymer is a chemically resistant shape memory polymer. The process includes uniaxially, biaxilally, or multiaxially shrinking the polymer by subjecting it to heat. Increasing the density and alignment of CNTs improves the conductivity of the assembly for strain gauge sensors and other applications that use CNTs. Other applications include batteries and chemical sensors.

Biaxial or multiaxially shrinkage of a CNT thin film produces wrinkled structures. As noted above, shrinking of metal films can produce wrinkling in the film. More generally, this wrinkling occurs if stiffness mismatch is provided between a substrate layer and a layer to be wrinkled or crumpled. We have found that a CNT thin film also produces wrinkling. It is believed that the total amount of van der Waals force between each individual CNTs is strong enough to create a stiff thin layer consequently wrinkling after biaxial or multiaxial shrinkage. This wrinkling phenomenon can be produced on shape memory polymers that shrink. We have also shown that the CNT thin film can be transferred onto a soft silicone substrate after removal of the shape memory polymer.

In some embodiments, the thin film of CNTs is shrunk by heating to a temperature of about 50° C., about 60° C., about 70° C., about 80° C., about 90° C., about 100° C., about 110° C., about 120° C., about 130° C., about 140° C., about 150° C., about 160° C., about 170° C., about 180° C., about 190° C., about 200° C., about 210° C., about 220° C., about 230° C., about 240° C., or about 250° C. or a range bounded by any two of the preceding numerical values.

A polyolefin is any of a class of polymers produced from a simple olefin (also called an alkene with the general formula CₙH₂ₙ) as a monomer. For example, polyethylene is the polyolefin produced by polymerizing the olefin ethylene. An equivalent term is polyalkene.

In some embodiments, the CNTs are dispersed in a solution of an organic solvent, such as chloroform, prior to deposition on a shape memory polymer. Other non-limiting examples of organic solvents include benzene, toluene and phenyl ethyl alcohol or other solvents (Li et al. 2012 “Dispersion of Carbon Nanotubes in Organic Solvents Initiated by Hydrogen Bonding Interactions” AIChE Journal 58: 2997-3002; Dumonteil et al. 2006 “Dispersion of carbon nanotubes using organic solvents” J Nanosci Nanotechnol 6(5): 1315-1318; and Ausman et al. 2000 “Organic Solvent Dispersions of Single-Walled Carbon Nanotubes: Toward Solutions of Pristine Nanotubes” J Phys Chem B 104: 8911-8915).

Densifying CNTs in a sensor application increases the sensitivity of the sensor, proportional to the degree to which a shape memory polymer shrinks. For example, a 95% reduction in area by shrinking on a polyolefin enables a much higher responsiveness. In some embodiments, a sensor containing densified CNTs, has a correspondingly lower electrical resistance upon densification of the CNTs. In some embodiments, the resistance of a film upon densification is reduced to about 100 kΩ In some embodiments, the resistance of a film upon densification is reduced to about 10 kΩ, about 50 kΩ, about 100 kΩ, about 150 kΩ, about 200 kΩ, about 250 kΩ, about 300 kΩ, about 350 kΩ, about 400 kΩ, about 450 kΩ, about 500 kΩ, about 550 kΩ, about 600 kΩ, about 650 kΩ, about 700 kΩ, about 750 kΩ, about 800 kΩ, about 850 kΩ, about 900 kΩ, about 950 kΩ, about 1000 kΩ, about 1100 kΩ, about 1200 kΩ, about 1300 kΩ, about 1400 kΩ or about 1500 kΩ or a range bounded by any two of the preceding numerical values. A low resistance film allows the development of highly sensitive devices that were previously not feasible based on previously existing technologies.

In some embodiments, the density amplification of the CNTs relative to an initial density upon application of the CNTs to a shape memory polymer is an increase of about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, about 1000%, about 1100%, about 1200%, about 1300%, about 1400% or about 1500% or a range bounded by any two of the preceding numerical values.

CNT density can be measured by a light transmittance test. In some embodiments, the CNT density results in light transmittance values of between about 30 to about 90%. In some embodiments the light transmittance is about 30%, about 35%, about 40%, about 45%, about 50%, about 55%, about 60%, about 65%, about 70%, about 75%, about 80%, about 85% or about 90% or a range bounded by any two of the preceding numerical values.

### Water-Soluble Polymers

One class of the water-soluble polymers includes polymers that are attached to a substrate through covalent bonds. Water-soluble polymers are generally chain-structured, non-crosslinked polymers having a hydrophilic group such as —OH, —CONH₂, —COOH, —NH₂, —COO, —SO₃, and —NR₃⁺ where R is alkyl or hydrogen.

Examples of natural water-soluble polymers include:

1) Starches, e.g., carboxymethyl starch, dialdehyde starch,

2) Celluloses, such as CMC, MC, HEC, HPC,

3) Tannins and lignins,

4) Polysaccharides, such as alginic acid, gum arabic, gum guar, gum tragacanth, and tamarind,

5) Proteins, such as gelatin, casein, glue and collagen,

Examples of synthetic water-soluble polymers include:

1) polyvinyl alcohol (PVA),
2) Plyethylene oxides, such as polyethylene oxide andpolyethylene glycol,
3) Acrylates, such as sodium polyacrylate,
4) Maleic anhydride polymers, such as methyl vinyl ether-maleic anhydride copolymers,
5) Phthalates, such as polyhydroxy ethyl phthalates,
6) Water-soluble polyesters, such as polydimethylol propionate,
7) Ketone aldehyde resins, such as methyl isopropyl ketone formaldehyde resin,
8) Acrylamides, such as polyacrylamide,
9) Polyvinyl pyrrolidone (PVP),
10) Polyamines, such as polyethylene imine,
11) Poly-electrolytes, such as polystyrene sulfonate, and
12) Others, such as water-soluble nylon.

Derivatives of these polymers are not limited to water-soluble ones, but may be of any form so long as they have, as a basic structure, the water-soluble polymer as mentioned above. Insolubilized derivatives may also be employed so long as they have freedom in molecular chain and can be hydrated.

Examples include esterified polymers, salts, amides, anhydrides, halides, ethers, hydrolyzates, acetals, formals, alkylols, quaternary polymers, diazos, hydrazides, sulfonates, nitrates, and ion complexes which are obtained by condensation, addition, substitution, oxidation or reduction reaction of the above-mentioned water-soluble polymers. Also included are polymers crosslinked with substances having more than one reactive functional group such as diazonium group, azide group, isocyanate group, acid chloride group, acid anhydride group, imino carbonate group, amino group, carboxyl group, epoxy group, hydroxyl group, and aldehyde group. Also included are copolymers with vinyl compounds, acrylic acid, methacrylic acid, diene compounds, and maleic anhydride.

Other examples of the water-soluble polymers are attached to the substrate through ionic bonds.

Typical examples of the water-soluble polymers include, in addition to polyvinyl pyrrolidone, carboxylates, sulfonates, and ammonium salts of the above-listed water-soluble polymers. Examples of the carboxylates include a sodium salt of methyl vinyl ether-maleic anhydride, sodium polyacrylate, polyacrylamide hydrolyzate, sodium carboxymethyl cellolose, and sodium alginate; examples of the sulfonates include sodium polystyrenesulfonate and sodium polyvinylsulfonate; and examples of the ammonium salts include ammonium salts of methyl vinyl ether-maleic anhydride and quaternized polyacrylamide.

In some embodiments, the substrate has, at least on its surface, a reactive functional group, which is covalently bonded with a water-soluble polymer or derivative thereof. The reactive functional groups existing on or introduced in the substrate surface are not particularly limited as long as they are reactive, bondable and cross-linkable with the water-soluble high polymers to affix the same. Examples of reactive functional groups include a diazonium group, azide group, isocyanate group, acid chloride group, acid anhydride group, iminocarbonate group, amino group, carboxyl group, epoxy group, hydroxyl group, and aldehyde group.

A shrink-induced high surface area electrochemical sensor may have a hydrophilic water-soluble polymer coating that completely fills invaginated pockets up until the time of contact with an aqueous sample to be measured. Alternatively, the invaginated pockets of the electrochemical sensor may only have a coating of PVP in the wrinkles, but not completely filling wrinkle invaginations. In either scenario, the hydrophilic coating provides an aqueous sample solution access to the complete surface of the sensor, including to depths of the invaginations.

### Example 1

### Superwetting and Aptamer Functionalized Shrink-Induced High Surface Area Electrochemical Sensors

Electrochemical sensing is moving to the forefront of point-of-care and wearable molecular sensing technologies due to the ability to miniaturize the required equipment, a critical advantage over optical methods in this field. Electrochemical sensors that employ roughness to increase their microscopic surface area offer a strategy to combatting the loss in signal associated with the loss of macroscopic surface area upon miniaturization. A simple, low-cost method of creating such roughness has emerged with the development of shrink-induced high surface area electrodes. Building on this approach, we demonstrate here a greater than 12-fold enhancement in electrochemically active surface area over conventional electrodes of equivalent on-chip footprint areas (Hauke et al. 2017 Biosensors and Bioelectronics 94: 438-442). This two-fold improvement on previous performance is obtained via the creation of a superwetting surface condition facilitated by a dissolvable polymer coating. As a test bed to illustrate the utility of this approach, we further show that electrochemical aptamer-based sensors exhibit exceptional signal strength (signal-to-noise) and excellent signal gain (relative change in signal upon target binding) when deployed on these shrink electrodes. Indeed, the observed 330% gain we observe for a kanamycin sensor is 2-fold greater than that seen on planar gold electrodes.

### Design

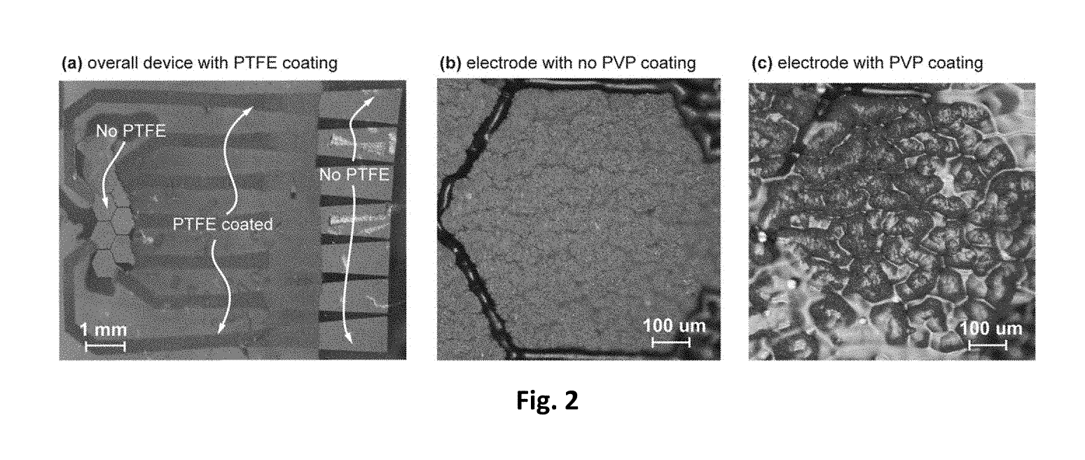

Devices were designed to have seven hexagonal electrodes with 360 μm side lengths, and were packed in a two row array spaced 50 μm apart. 300 μm wide trace connections were routed together with 1000 μm pitch. FIG. 2a provides a picture of a completed device with these dimensions. A Mylar photomask of the design was drafted (AutoCAD 2015) and printed by CAD/Art Services, Inc.

### Bare-Gold Device Fabrication (for EASA Measurement)

Shrink electrode fabrication closely followed the procedures as those of previous work (Pegan, J. D., Ho, A. Y., Bachman, M., Khine, M., 2013. Flexible shrink-induced high surface area electrodes for electrochemiluminescent sensing. Lab Chip 13, 4205-4209). Polyolefin films (0.5 mil seven layer polyethylene/polypropylene blend, Sealed Air Corp.) were cut into 70 mm×70 mm squares and adhered to silicon wafer substrates using 70% ethanol and Kapton tape to seal the edges. 40 nm of gold was then sputter deposited (Q150R S, Quorum Technologies) onto each sample. The samples were subsequently photopatterned using Shipley 1827 (Microchem) positive resist followed by UV exposure (MA6, Suss Microtec) through a photomask. After development, the wafers were wet etched in I₂:KI:H₂O (1:4:40) gold etchant to remove the areas of gold that were exposed through the photolithographic patterning. Finally, acetone was used to remove the remaining photoresist. Once patterned, the PO films were carefully removed from the silicon wafer backing.

Polytetrafluoroethylene (PTFE) was added to electrically insulate the long gold electrodes leading to the hexagonal gold sensor surfaces (FIG. 2a). This same PTFE coating also prevented wicking of fluid along the electrode leads (and therefore ensured that the tested sensor area is constant during testing). To achieve this coating, fluoropel solvent (Cytonix PFC-160) with mass ratio of 0.1% Teflon AF1600 (Dupont) was applied to electrical trace areas using a cotton swab. After allowing the solvent to dry for 30 min the devices were then shrunk by oven baking at 150° C. for 5 min (Gabardo, C., Zhu, Y., Soleymani, L., Moran-Mirabal, J. M., 2013. Bench-top fabrication of hierarchically structured high surface-area electrodes. Adv. Funct. Mater. 23, 3030-3039; Pegan, J. D., Ho, A. Y., Bachman, M., Khine, M., 2013. Flexible shrink-induced high surface area electrodes for electrochemiluminescent sensing. Lab Chip 13, 4205-4209).

### Polyvinyl Pyrolidone Superwetting Device Fabrication and Characterization

Aqueous solution with a mass ratio of 6% polyvinyl pyrolidone (Sigma-Aldrich PVP10) and 0.01% non-polar surfactant (Triton X100) was prepared by stirring for 5 h. Approximately 2 μL of solution was applied to each exposed electrode area using a pipet such that the surface was nearly covered. The device was immediately placed in vacuum (Thermo Scientific Lindberg Blue M) at 27 in. Hg (gauge) for approximately 5 min until noted air bubble formation on the meniscus of PVP solution was observed to cease. Excess solution was then hand removed by wicking into a paper towel. The result was an even coating of clear PVP with all of the wrinkles filled without gas pockets, as seen by comparing FIGS. 2b and c.

A three electrode electrochemical cell with a Pt wire counter (BASi MW-1032) and Ag/AgCl reference electrode (eDAQ ET072) was used to obtain cyclic voltammagrams in 0.1 M H2SO4 (Fisher Scientific H₂SO₄). PVP coated shrink electrodes were compared to a commercial gold disk electrode (eDAQ ET076) as working electrodes in the cell. Cyclic voltammetry (CV) scans were run from −0.2 V to 1.7 V at a scan rate of 20 mV/s (PalmSens3). The reduction peaks of the fourth CV scans were integrated using PSTrace 4.6 software. Reduction peak integrals of the CV scans were converted into power densities by dividing by the given electrodes footprint area. Gain in EASA was determined by the ratio of the power densities of PVP coated shrink electrodes to the bulk gold.

### E-AB Sensor Fabrication and Characterization

Electrodes were immersed in 0.5 M sulfuric acid and pulsed between 0.0 V and 2.0 V vs Ag/AgCl for 400,000 cycles using the chronoamperometry technique. Each pulse lasted for 2 ms with no quiet time. This standard preparation procedure was performed to clean the gold surface as well as further roughen it to facilitate coverage by the DNA probes. All probe oligonucleotides, 5′-HO—(CH₂)₆—S—S—(CH₂)₆-GGGACTTGGTTTAGGTAATGAGTCCC-(CH₂)₇-methylene blue-3′ (SEQ ID NO: 1), were dissolved with Tris buffer to a final concentration of 100 μM, aliquoted and stored at −20° C. ready for use. To fabricate our sensors, the freshly cleaned electrodes were immersed in 200 nM DNA solution for 1 h at room temperature, which was previously prepared by incubating a solution of 100 μM thiolated DNA and 20 mM TCEP (tris-(2-carboxyethyl)) phosphine hydrochloride (1:200) for 1 h at room temperature, and further diluted by PBS buffer (pH 7.0). The resulting sensors were washed with deionized water and then incubated in 20 mM 6-mercaptohexanol solution overnight at 4° C. The functionalized sensors were then rinsed with pure water prior to use. Electrochemical measurements were performed at room temperature using a CHI630C potentiostat with a CHI684 Multiplexer (CH Instruments, Austin, Tex.) and a standard three-electrode cell containing a Pt counter electrode and a Ag/AgCl (3 M NaCl) reference electrode. Square wave voltammetry was performed using a potential window of −0.10 V to −0.4 V (versus Ag/AgCl), potential step of 1 mV, and amplitude of 0.05 V at a frequency of 500 Hz. Curve fitting of data was performed using Microsoft Excel software.

### Device Operation Theory

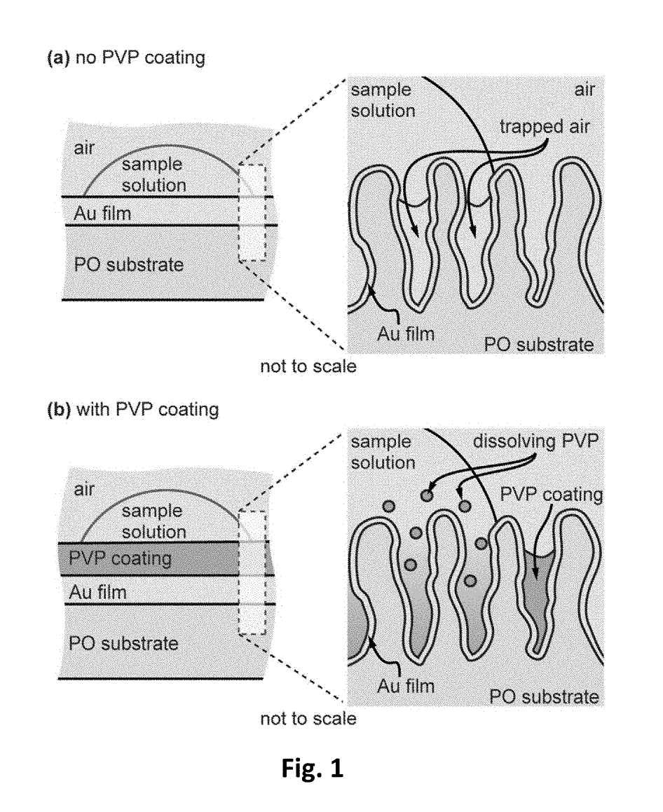

Deep areas of the shrunken surface trap air very efficiently when interfacing aqueous liquids (FIG. 1a). As air does not readily dissolve into water, this structure diminishes the total EASA. Others have leveraged this property as an advantage in developing antibacterial surfaces (Freschauf, L. R., McLane, J., Sharma, H., Khine, M., 2012. Shrink-induced superhydrophobic and antibacterial surfaces in consumer plastics. PLoS One 7, e40987), but it is a major complication for electrochemical sensing applications and the limiting factor for the EASA improvements. FIG. 1b depicts the proposed superwetting enhancement by simply replacing the air and filling the wrinkles with PVP. In this scenario, aqueous sample is incident on a hydrophilic surface that it can fully wet against. As PVP dissolves, it diffuses into the bulk solution while the sample in turn flows in to fill the space left by the PVP. This modification allows sample to access more electrode surface and maximize the EASA gain achieved by shrinking.

For validation, E-AB sensors were also utilized (Biagiotti, V., Porchetta, A., Desiderati, S., Plaxco, K. W., Palleschi, G., Ricci, F., 2012. Probe accessibility effects on the performance of electrochemical biosensors employing DNA monolayers. Anal. Bioanal. Chem. 402, 413-421), which can work continuously even in whole blood. E-AB sensors are based on DNA folding that occurs when an analyte is bound. The folding event then changes the distance between a redox couple tag and a gold electrode surface, which changes the amount of charge transfer between the redox couple and the electrode (Pheeny, C. G., Barton, J. K., 2012. DNA electrochemistry with tethered methylene blue. Langmuir 28, 7063-7070). Mechanisms for charge transfer are localized at the surface of the electrode, allowing E-AB sensors to operate with low susceptibility to surface fouling and without reagents (Heikenfeld, J., 2016. Non-invasive analyte access and sensing through eccrine sweat: challenges and outlook circa 2016. Electroanalysis 28, 1242-1249).

### Results

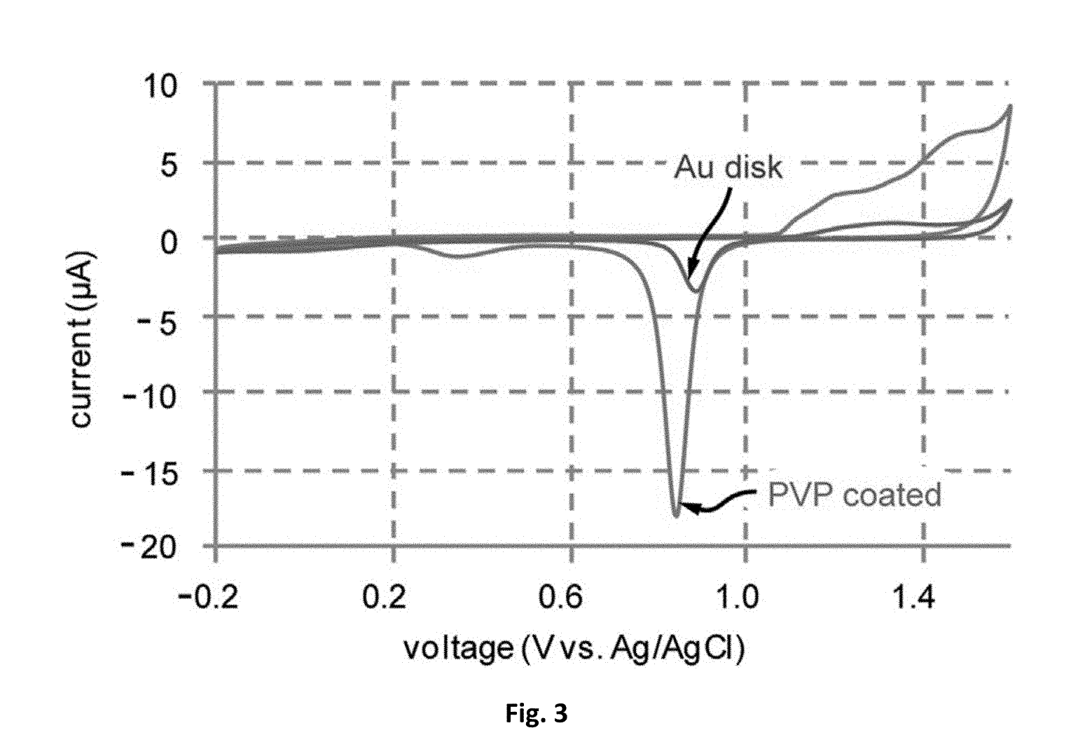

Footprint areas for the gold disk and polyvinyl pyrolidone (PVP) coated shrink electrodes were measured to be 0.79 mm² and 0.31 mm² respectively. CV curves for these electrodes are shown in FIG. 3. Using the CV data and measured areas, the integrated reduction peak power densities were determined to be 0.367 μA V/mm² and 4.67 μA V/mm² respectively, indicating a 12.7-fold enhancement in EASA with the PVP coated shrink electrode. The result increases upon the previous 6.6-fold result observed (Pegan, J. D., Ho, A. Y., Bachman, M., Khine, M., 2013. Flexible shrink-induced high surface area electrodes for electrochemiluminescent sensing. Lab Chip 13, 4205-4209.) by a factor of 1.9. This greater than 12-fold gain was repeatable. To verify the results, two separate control CV scans were run using the gold disk electrode. One was run in 0.1 M H₂SO₄ solution and the other in bulk 6% PVP, 0.01% Triton X100 solution used to coat the shrink electrodes. The former produced a voltammagram characteristic of H₂SO₄ while the pure coating voltammagram showed insignificant redox activity in the H₂SO₄ reduction peak range around 0.85 V, indicating that the amplification from the PVP coated shrink electrodes was due to the increased wetting on the surface and not a response to the dissolved coating in the sample.

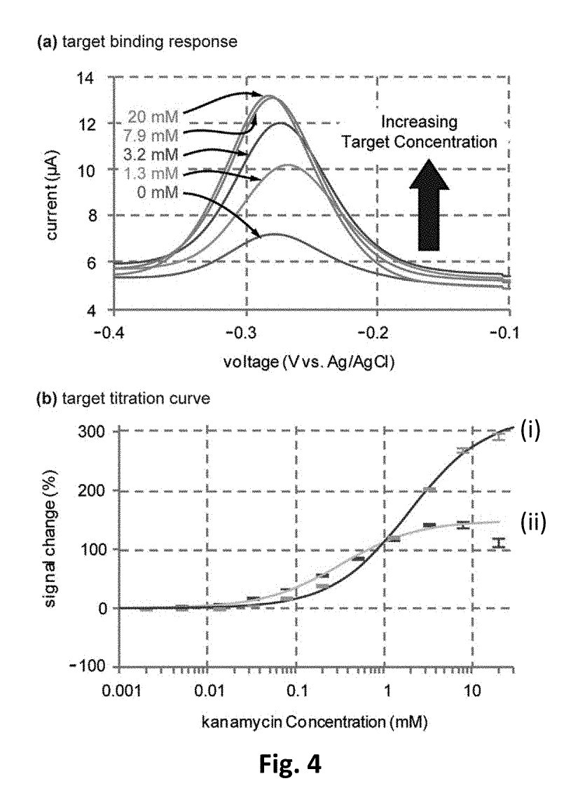

Square wave voltammagrams for the E-AB sensor are shown in FIG. 4a. As expected, the methylene blue reduction peak at −0.28 V increases significantly with increasing kanamycin concentrations. The same data presented as a target titration curve produces the monotonic Langmuir isotherm behavior expected for this type of binding event, yielding a dissociation constant of 2.0 mM (FIG. 4b). Also of note, the plot (curve i) shows the 330% signal change seen upon saturating target represents a >2-fold improvement over that seen for the same E-AB sensor when fabricated on a traditional, macroscopic gold electrode (FIG. 4b, curve ii). Therefore, in addition to a total signal increase due to increase in surface area, the signal change (response) of the E-AB sensor has also increased. The resulting increase in sensitivity (to small changes in target concentration) for the shrink electrode is easily seen in the difference in slopes of the curves in their pseudo-linear regions. The detection capability is within the 2-6 μM range that is clinically relevant for aminoglycoside concentrations (Rowe, A. A., Miller, E. A., Plaxco, K. W., 2010. Reagentless measurement of aminoglycoside antibiotics in blood serum via an electrochemical, ribonucleic acid aptamer-based biosensor. Anal. Chem. 82, 7090-7095; Setia, U., Gross, P. A., 1976. Administration of tobramycin and gentamicin by the intravenous route every 6 h in patients with normal renal function. J. Infect. Dis. 134, S125-129.). An equilibration time of 2 min (time to achieve 90% of maximum signal) was observed at 1 mM target concentrations. This is slightly slower than the equilibration kinetics seen with planar electrodes, presumably due to slower diffusion associated with the greater topological complexity of the wrinkled surface.

It should be noted that the self-assembled monolayer (SAM) of thiol required for E-AB sensing presented a very effective superwetting modification by itself. In fact, sample solution uncontrollably wet over all areas of the completed devices. Therefore the PTFE coating was added to confine the sample solution to the electrode areas of the devices and no dissolvable polymer was necessary for superwetting of the surface for the E-AB sensors.

### DISCUSSION

The PVP coating superwetting strategy produces a nearly two-fold increase in EASA over shrinking alone. However, the 12.7-fold increase in current seen when shifting from a planar electrode to a PVP coated shrink electrode still indicates that there is potential for further enhancement toward the 20-fold theoretical maximum. Further optimizations of this process may lead to even better results. For instance, PVP can be replaced by a number of dissolvable polymers, such as poly-vinyl alcohol or sucrose. The choice of coating is ultimately application specific to the end user based on compatibility with intended detection target and other sample constituents. Another optimization strategy could be minimization of polymer film thickness which could aid the sample solution in entering more confined areas of the wrinkled surface more rapidly. Small volume samples could also benefit from this by limiting the possibilities of interference from a relatively large amount of PVP as compared to target solution sample.

Photolithography was used to produce the sputter mask for gold patterning in this instance, due to the close packing of the electrode array. It should be noted that this is not necessary in all applications and could be simplified by use of shadow masks that can be patterned using a variety of desired methods. Another fabrication process improvement would be to develop a pre-shrinking PVP coating method. This change would eliminate the vacuum degas step and improve the manufacturing scalability for future commercial development. Thus, Triton X100 would also no longer be required as this surfactant was added solely to assist the PVP solution in wetting to the gold surface in the presence of trapped air. This method is feasible judging from the PTFE coating step which showed no observable effects on the final shrunk micro-scale surface topography upon visual inspection. However, more advanced inspection techniques such as microscopy imaging or electrochemical methods are beneficial to verify that the nano-scale secondary structuring is not adversely affected.

The 330% signal gain seen for E-AB kanamycin sensors built on shrink electrodes is significantly greater than that seen on planar electrodes, which is more typically only 150% (FIG. 4b). Higher gain generally translates into improved measurement precision as it enables robust detection at lower fractional receptor occupancy. The origins of this higher gain are unclear, as the enhanced surface area (and thus signaling current) produced by nanoscale texturing of the surface should enhance the current produced by both the bound and unbound states of the aptamer receptor, leading to no net change in relative gain. Further research is will provide a greater understanding of the observed higher gain. In contrast to the improved gain, the binding kinetics of this E-AB sensor were slightly slower, requiring 2 min to reach ˜90% maximum signal, while simple, planar electrodes typically require less than 1 min to achieve this mark. This is a likely product of restricted advective and diffusive transport in wrinkled structures. However, this drawback is a comparably minor tradeoff with respect to the signal gain enhancement. Whether optimizing the shrink E-AB sensor fabrication parameters could further extend the signal gain as well as boost other performance metrics such as sensitivity and limits of detection remain open questions; the results reported here are offered as a proof-of-concept for the validity and possible advantages of using modified shrink electrodes in sensors of this class (Lubin, A. A., Plaxco, K. P., 2010. Folding-based electrochemical biosensors: the case for responsive nucleic acid architectures. Acc. Chem. Res. 43, 496-505.). These results demonstrate the usefulness of using aptamer probes.

### CONCLUSIONS

Significant improvements were achieved upon the already impressive previously published shrink electrode performance. A 12.7-fold EASA enhancement was achieved with the addition of a dissolvable polymer coating. This enhancement doubles the previous benchmark and can be done with one additional simple fabrication step (PVP coating). Additionally the shrink electrodes far surpassed expectations as an E-AB sensor platform. Its 330% signal gain is a 2.2-fold improvement upon previously reported results for similar aminoglycoside E-AB sensors recorded using more traditional “flat” electrodes. These advancements further support the candidacy of shrink electrodes for robust, easily fabricated, and low-cost electrochemical sensor solutions in lab-on-chip and point-of-care applications.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

All figures, tables, and appendices, as well as patents, applications, and publications, referred to above, are hereby incorporated by reference.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

What is claimed is:

1. A sensor comprising:
a flexible substrate,
a conductor disposed on the flexible substrate, and
a hydrophilic surface coating disposed on the conductor,
wherein the flexible substrate and the conductor form wrinkles as a result of the substrate being shrunk, and wherein the hydrophilic surface coating fills the wrinkles or covers surface areas of the conductor within invaginations of the wrinkles.

2. The sensor according to claim 1, wherein the flexible substrate is a polyolefin film.

3. The sensor according to claim 1, wherein the conductor is a metal.

4. The sensor according to claim 3, wherein the metal is gold.

5. The sensor according to claim 1, wherein the conductor comprises one-dimensional (1D) nanostructures.

6. The sensor according to claim 5, wherein the one-dimensional (1D) nanostructures are nanotubes.

7. The sensor according to claim 1, wherein the hydrophilic surface coating is a hydrophilic polymer.

8. The sensor according to claim 7, wherein the hydrophilic surface coating is a natural aqueous soluble polymer.

9. The sensor according to claim 8, wherein the naturally occurring aqueous soluble polymer is a protein.

10. The sensor according to claim 7, wherein the hydrophilic surface coating is a synthetic aqueous soluble polymer.

11. The sensor according to claim 10, wherein the synthetic aqueous soluble polymer is polyvinyl pyrolidone (PVP).

12. The sensor according to claim 1, further comprising an aptamer bound to the sensor.

13. The sensor according to claim 12, wherein the aptamer is specific to an aminoglycoside antibiotic.

14. The sensor according to claim 13, wherein the aptamer is specific to kanamycin.

15. The sensor according to claim 14, wherein the aptamer is SEQ ID NO: 1.

16. The sensor according to claim 1, wherein the wrinkles comprise micro-scale invaginations.

17. A method of preparing the sensor according to claim 1, comprising:
obtaining a flexible substrate,
depositing a conductive layer onto the flexible substrate,
shrinking the flexible substrate with the deposited conductive layer, wherein the flexible substrate and the deposited conductive layer forms wrinkles; and
filling the wrinkles formed by the flexible substrate and the conductive layer with a layer of an aqueous-soluble polymer.

18. The method according to claim 17, wherein the aqueous-soluble polymer is applied to the shrunken flexible substrate in an aqueous solution comprising the aqueous-soluble polymer and a surfactant.

19. The method according to claim 17, wherein the shrunken flexible substrate is placed in a vacuum to remove air bubbles.

20. A method of detecting an amount of an analyte in an aqueous solution, the method comprising:
obtaining a sensor according to claim 1,
contacting the sensor with the aqueous solution, and
detecting an electrical signal with the sensor, wherein the electrical signal is indicative of the amount of the analyte.


SEQUENCE LISTING (1 sequence)

SEQ ID NO: 1 - DNA, 28 bp, Artificial Sequence
  oligonucelotide probe
  misc_structure (1)..(1): substituent attached to 5′ end HO-(CH2)6-S-S- (CH2)6-
  misc_structure (28)..(28): substituent attached to 3′ end -(CH2)7- methylene blue
ngggacttggtttaggtaatgagtcccn